(19) United States
(12) Patent Application Publication
(10) Pub. No.: US 2009/0118698 A1
(43) Pub. Date: May 7, 2009

(54) DEVICE AND METHOD FOR APPLYING AN ACTIVE INGREDIENT TO THE SKIN

(75) Inventor: Horst Liebl, Zelsheim (FR)

Correspondence Address: ROBERTS MLOTKOWSKI SAFRAN & COLE, P.C.;Intellectual Property Department, P.O. Box 10064, MCLEAN, VA, 22102-8064, US

(73) Assignee: Edelgard Liebl, Zelsheim/Alsace (FR)

(21) Appl. No.: 11/721,627

(22) PCT Filed: Dec. 14, 2001
(86) PCT No.: PCT/EP01/14747

§ 371 Date: Jun. 13, 2007

(30) Foreign Application Priority Data
Dec. 20, 2000 (DE) 100 63 634.9

(51) Int. Cl. A61M 5/32

(52) U.S. Cl. 604/506; 604/173

(57) ABSTRACT

The invention relates to a device and a process for introducing an active ingredient into the skin. The device comprises a roller that is mounted to rotate around the longitudinal axis thereof and on whose outside peripheral surface a number of needles project radially outward. After the active ingredient has been applied on the skin, the roller rolls over the skin. In this case, the needles penetrate the skin and open up fine channels there, through which the active ingredient penetrates through the epidermis up to the dermis.

The invention relates to a device as well as a process for introducing an active ingredient into the skin.

In dermatology and cosmetics, the introduction of active ingredients into the skin is of great importance in, for example, preventing the aging of the skin and revitalizing the latter. With increasing age, the thickness of the skin layers decreases. Fatty tissue in deeper skin layers is broken down, and the collagen fibers shrink and harden. The skin dries out, loses its transparency and thus its fresh, satiny appearance.

A number of active ingredients are known that can counteract the aging of the skin and that promote the revitalization of the skin. These active ingredients are usually applied on the skin in the form of creams. The problem consists in that, however, only a small proportion of the active ingredients actually penetrates those regions of the skin in which they can exert their action.

In the structure of the skin, a distinction is made basically between dermis and epidermis. In the lower dermis, those processes take place that contribute to the new structure and to the revitalization of the skin. In the dermis, the formation of new collagen tissue is carried out. So that active ingredients can exert their full action in the regeneration of the skin, it is necessary to convey the latter up to the dermis. The outer skin layers, however, work against the epidermis. The epidermis is usually divided into 5 skin layers and primarily consists of dead horny cells that are constantly replaced by the organism, so that the epidermis is constantly renewed. Depending on the age and region of the body, the epidermis has a thickness of about 0.03 to 0.1 mm. The epidermis forms a type of protective shell for the lower skin layers against outside influences such as bacteria, dirt, etc. It also prevents the penetration of substances that are useful to the skin, however.

Clinical studies have shown that active ingredients applied in the conventional way on the skin remain on more than 95% of the epidermis. Only at most 5% of the active substances penetrate the epidermis and go into the dermis, where they can exert their desired action.

To increase the proportion of active substances that penetrate the dermis, the proportion of active ingredient in the creams, gels, etc., is often increased. Thus, primarily also the concentration of the active ingredients in the epidermis is increased, however, where a majority of the substances remains. Often, side effects such as the destruction of the epidermis cell layers result therefrom with an associated oversensitivity of the skin to sunlight (hyperpigmentation). Moreover, creams with a high proportion of active ingredients are unnecessarily expensive. Another possibility to increase the proportion of the active ingredients that penetrate the dermis exists in the addition of adjuvants such as liposomes or acids. Also, these additives result, however, in the destruction of the epidermis layer and have the same disadvantages that were described above.

Another procedure consists in removing the epidermis mechanically to facilitate the active ingredients' access to the dermis. For example, the abrading of the upper skin layers with diamond milling as well as the evaporation of the skin with a laser are common. Also, in this case, the skin is stripped of its important protective layer, in which the melanocytes are produced, which protect the skin from harmful ultraviolet effects. As a result thereof, the thus treated skin is greatly reddened over months and is extraordinarily light-sensitive.

In view of this, the object of the invention is to indicate a process and a device, with which in the case of the fewest possible side effects without long-term damage of the epidermis, it is possible to introduce active ingredients into the skin that can exert their full action in the case of very low concentration.

The achievement of this object is possible with the device according to claim 1 as well as the process according to claim 20. Preferred embodiments can be derived from the subclaims.

In a first aspect, the invention relates to a device for introducing an active ingredient into the skin. This device comprises a roller that is mounted to rotate around the longitudinal axis thereof, on whose outside peripheral surface a number of needles project radially outward.

In the process according to the invention, this device is used to introduce an active ingredient into the skin. For this purpose, the active ingredient or a mixture that contains the active ingredient is applied on the skin. Then, the device is guided over the skin so that the needles that project over the outside peripheral surface thereof penetrate the skin. By the needles penetrating the skin, minute channels are opened, through which the applied active ingredient is conveyed into the deeper skin layers. Since the channels in the skin remain open for a long time, additional active ingredient can penetrate the skin. This process can optionally be supported by massaging or similar measures. Depending on the frequency of the movement of the device according to the invention over the skin, the number of puncture channels can be controlled specifically and thus also the degree to which active ingredient can penetrate the treated skin. Since the puncture channels close again after a few minutes, the epidermis is damaged only minimally. The damage initially mentioned in connection with the removal of the epidermis therefore does not occur in the case of the invention. In any case, a slight reddening of the skin is observed that recedes completely, however, within a few days, in most cases within one or two days. The treatment of the skin according to the invention is therefore virtually free of side effects and generally completely pain-free. It is, moreover, simple and economical.

Another advantage consists in that the active ingredient concentration in the creams, gels, etc., that are used can be considerably reduced, since the insertion of the active ingredient into the lower skin layers is very effectively possible. Damaging the epidermis by increased active ingredient concentrations can be prevented. Previous studies with vitamin A preparations have shown that when using the device according to the invention and the process according to the invention, the active ingredient concentration can be reduced by about 80% compared to conventional creams or other active ingredient mixtures, but in this case at least equally good cosmetic results with simultaneously fewer side effects can be achieved.

In principle, the invention is suitable for any active ingredient already used to date in connection with the treatment of the skin. Those preparations that require collagen synthesis can be referenced only by way of example. Another possible application is the treatment of scar tissue.

The number of puncture channels in the skin can be set, on the one hand, by the frequency with which the roller of the device according to the invention is rolled over the skin. In addition, it is within the scope of the invention, of course, to control the number of puncture channels via the number of needles with which the rotatable roller is equipped. To achieve as uniform as possible a distribution of the puncture channels in the treated skin, it is advisable to distribute the needles over the outside peripheral surface of the roller uniformly. For example, the needles can be arranged in series that run parallel to the longitudinal axis of the roller.

In addition, it is useful that all needles with the same length project over the outside periphery of the roller, so that all puncture channels have essentially the same depth. The length with which the needles project over the outside peripheral surface—referred to below as the penetration depth of the needles—suitably depends on the intended use. Depending on the cosmetic or medicinal indication and depending on the type of skin parts to be treated, the needles may have a different penetration depth. Usually, the latter are in the range of 0.12 to 1.5 mm. For purely cosmetic treatments, the penetration depth is usually to be less than for medicinal treatments, in which it may be useful if the punctures extend into the dermis. In the latter cases, penetration depths of over 1.5 mm may also occasionally be necessary.

The treatment of the skin with a device according to the invention, whose needles have a penetration depth of 1 to 1.5 mm or longer, results in that puncture channels are formed that extend into the dermis. In the very fine wounds, the dermis reacts with the formation of new blood vessels (vascularization) and the formation of new collagen tissue. The formation of new collagen is thus promoted in two ways, namely, on the one hand, by the fine puncture channels and the thus induced fibrous reactions, and, on the other hand, by the active ingredients that penetrate the dermis via the channels (for example, Vitamin A).

Also, the sugar deposit on the collagen fibers is significantly mitigated by treatment with the device according to the invention. The collagen fibers that are hardened by the sugar are released by the sugar molecules, the collagen synthesis is stimulated, and the skin is once more elastic. When using longer needles, which penetrate the dermis, it may be necessary to anesthetize the skin. Corresponding agents, for example creams, are known for this purpose. Only a relatively short-term irritation of the skin can be observed, however, even with using long-needle devices according to the invention. The reddening that occurs has generally disappeared again within 3 to 4 days.

To facilitate the penetration of the needles into the skin, the latter are suitably designed at least in an area that projects over the outside peripheral surface of the roller that is designed outward and tapers to a point. Suitably, each needle point is as thin as possible, without, however, a breaking of the needles being expected. How large the diameter in the point area of the needle is depends primarily on the material that is used for the needle and the intended application. Suitable diameters for the point areas of the needles are between 0.05 and 0.1 mm, in particular 0.08 mm. In the remaining area of the needle, outside of the point area, diameters between 0.15 and 0.3 mm have proven suitable.

The needles of the device according to the invention preferably consist of metal and in particular high-grade steel. The points of the needles are usually beveled and then electropolished.

The roller, over which the needles of the device according to the invention project, suitably consists of plastic, and in particular a plastic that can be processed in the injection-molding process. In a preferred variant of the invention, the roller is built up from several parallel disks, between which the needles are embedded. In this case, it is preferred to embed the needles in recesses that extend outwardly at least on one of the sides of the disks in radial direction. To prevent a slipping of the needles inside the roller, a stop on the inside end of the recesses is suitably provided for each of the needles.

The needles are preferably glued into the recesses on the disks. For this purpose, in principle any adhesive that is compatible with the plastic of the roller is suitable. Both the plastic of the roller as well as the adhesive should be dermatologically harmless. Moreover, both materials should be resistant to such radiation, which usually is used to sterilize medical devices. Plastics and adhesives that are resistant to ultraviolet and gamma rays are therefore preferred within the scope of the invention. In addition, it is suitable if both materials are resistant to common purification and sterilization liquids, such as aqueous hydrogen peroxide. As an example of a suitable adhesive, cyanoacrylate adhesive can be mentioned.

In the case of a roller that is built up from several disks, first the needles are glued into the recesses that are provided on the disks. Then, the disks are connected to one another in parallel to one another. Also, in this respect, an adhesive can be used. So that the disks have the desired orientation to one another, it is especially preferred within the scope of the invention to provide at least one centering projection on the surfaces of a disk. This at least one centering projection corresponds to a corresponding centering recess on the surface of the adjacent disk. It is especially suitable if the centering projection is used simultaneously as a rear stop for the needles. Especially preferred are those centering projections that simultaneously result in a centering of adjacent disks and in setting a specified angular offset of the needles of the adjacent disks to one another. Suitable, in this connection, for example, is a projection in the form of a gear rim, which projects into the center of the disk. A corresponding recess in the form of the gear rim is designed in an adjacent disk and snugly receives the projection of the other disk.

In this case, the arrangement of disks that are adjacent to one another can be carried out such that the needles of all adjacent disks lie on lines that run parallel to the longitudinal axis of the roller. As an alternative, it is possible to arrange the needles of adjacent disks in gaps.

The number of needles on a roller of specified size can be controlled, on the one hand, by the number of disks—i.e., by the disk thickness—as well as the number of needles on each disk. It has been shown that packing the roller too densely with needles can have a negative effect, since a kind of “fakir cushion effect” occurs and the needles can have difficulty penetrating the skin. Suitable numbers of needles are ensured if, for example, the needles on each disk are arranged in straight lines, which form an angle of 15 or 30° from the center of the disk. This corresponds to 24 or 12 needles on each disk. Suitable disk thicknesses are approximately, for example, 2 to 3 mm, in particular 2.5 mm.

The number of disks used suitably depends on the intended use of the device according to the invention. Depending on the size of the main portion to be processed, for example, 2 to 3 disks may be sufficient, but also 10 or more disks can be used. End disks can be used as suitable front closings of the disk arrangement that result in, for example, a smooth, front closing of the thus produced roller.

The diameter of the roller or the disks forming them can also be varied over a wide range. By varying the size of the diameter, influence on the distance of adjacent needles can also be exerted. In addition, it is possible to set the length of the supernatant of the needles over the outside periphery by variation of the size of the diameter and thus to control the penetration depth of the needles. In this way, without changing the needle length, devices with different penetration depths of the needles can be produced. Conversely, it is possible to keep the diameter of the roller or the disks that form the roller constant and, instead of this, to vary the needle length and to obtain in this way devices with different penetration depth of the needles. By way of example, roller diameters of 1 to 5 cm can be mentioned. Especially suitable are rollers with a diameter of about 2 to 3 cm.

Various possibilities are conceivable, in which way the rollers of the device according to the invention, equipped with needles, can be mounted to rotate. One possibility consists in fastening the roller in a rotatable manner to a fork, which shifts the stress on the roller in the longitudinal direction. In turn, a handle is arranged on the fork. In a preferred variant, the roller has a through-hole that comprises the longitudinal axis of the roller. In this through-hole, a shaft is inserted, on which in turn the fork is fastened to the handle. As an alternative, of course, it is also possible that the roller has lateral projections, which are fastened to rotate in the fork. Conversely, the fork can also have projections directed in the direction of the roller, which engage in the roller in front recesses, so that the roller can be rotated around the fork projections. The material selection for holding the roller is not further limited. By way of example, plastic or metal can be mentioned.

The invention is to be explained in more detail below based on a drawing. Therein:

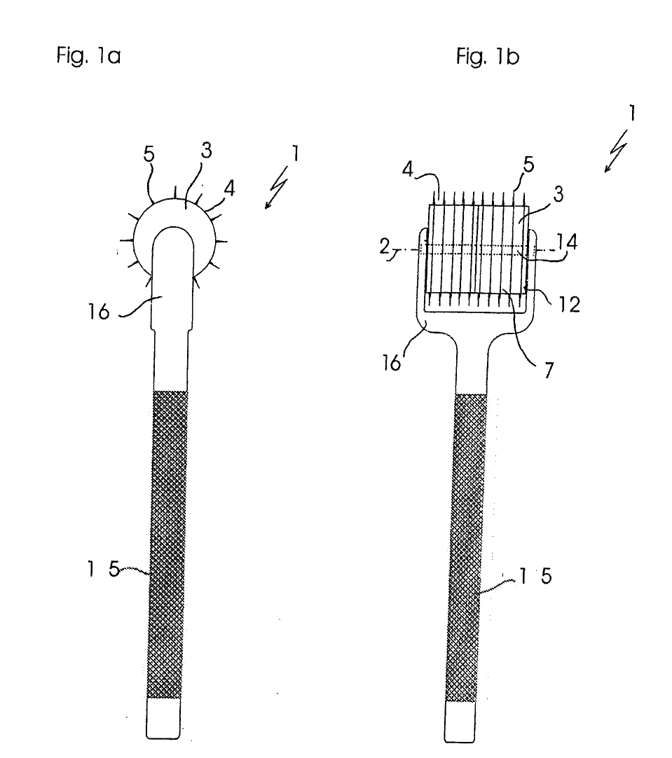

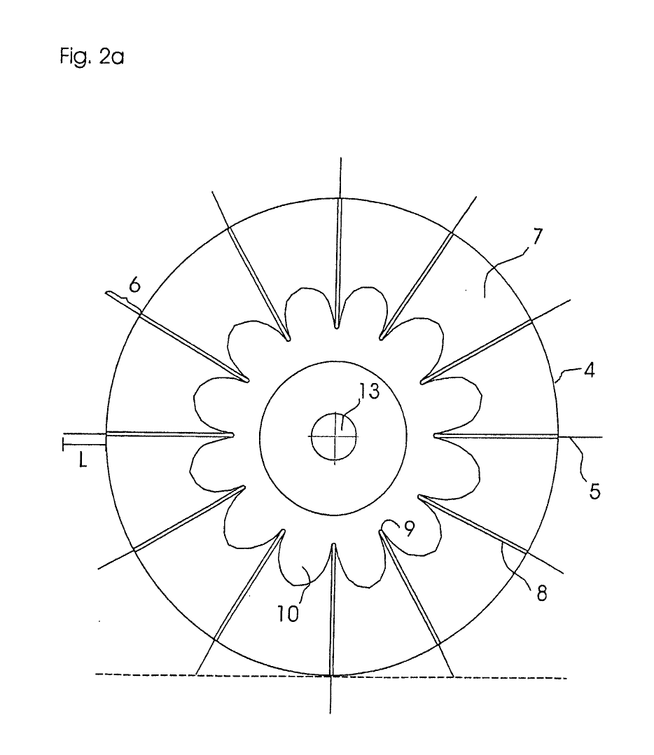

FIGS. 1a and b diagrammatically show a device according to the invention in two different side views;

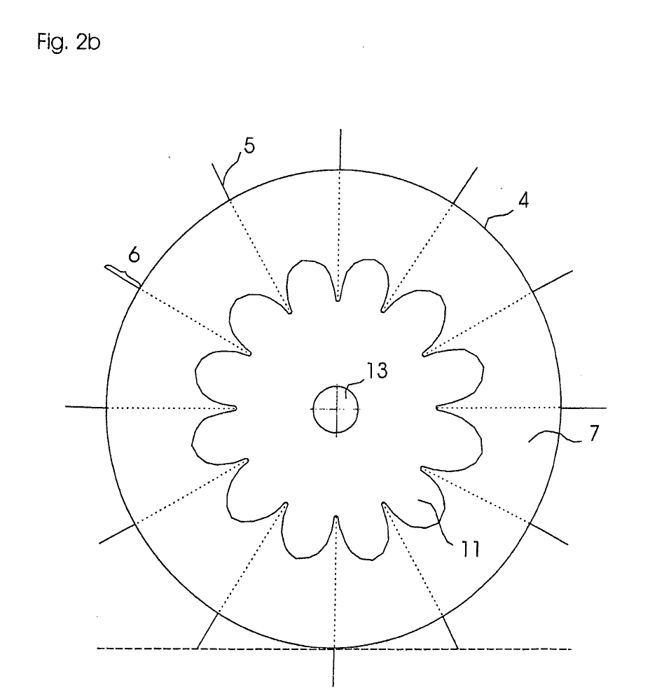

FIG. 2a diagrammatically shows a disk equipped with needles, which is a component of the device according to FIG. 1, in top view;

FIG. 2b diagrammatically shows a top view at the back of the disk shown in FIG. 2a, and

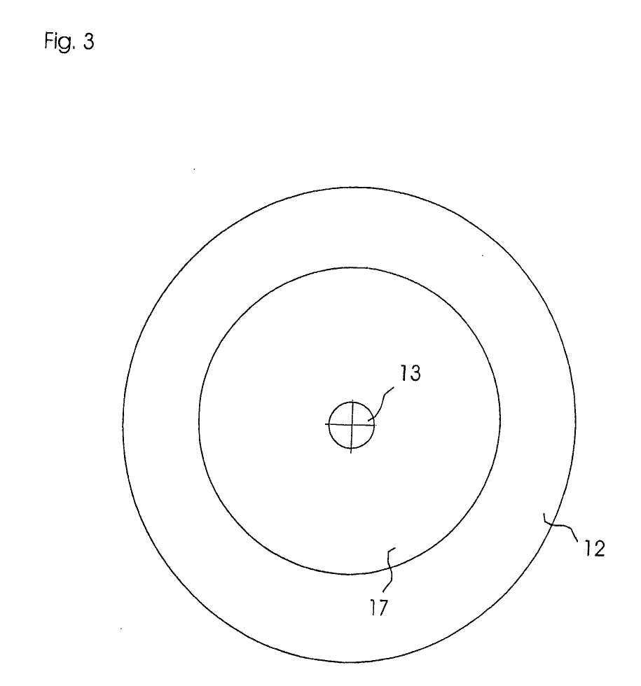

FIG. 3 diagrammatically shows a front end disk of the device shown in FIG. 1.

FIG. 1 shows in detail a device 1 according to the invention for introducing active ingredients into the skin in a perspective representation. The device essentially consists of a roller 3, which is mounted to rotate around the longitudinal axis 2 thereof on a fork 16 that is provided with a handle 15. FIG. 1a shows a side view on one of the front sides of roller 3; FIG. 1b rotates the device by 90°.

Via the outside peripheral surface 4 of the roller 3, needles 5 project radially outward at regular intervals. The needles taper outward to a point. To make the representation easier to see, the projecting points of the needles are depicted in overly enlarged form. In a preferred embodiment of the invention, the needles project about 0.12 to 1.5 mm over the outside peripheral surface 4 of the roller 3. The roller itself has, for example, a diameter of 2 cm. Its length is about 3 cm.

To treat the skin with the device according to the invention, an active ingredient or a mixture that contains the active ingredient is first applied on the skin. Then, the device according to the invention is placed on the skin with the needle-equipped roller 3, and the roller is rolled back and forth on the skin with light pressure. In this case, the roller rotates around the shaft 14, and the series of needles 5 penetrate the skin in succession and can produce narrow puncture channels there. The active ingredient can pass through the epidermis via these puncture channels and can extend into the dermis, where it can exert its desired action.

FIGS. 2 and 3 describe the components from which the roller 3 that is equipped with needles 5 is built up.

The roller 3 consists of several disks, which are arranged parallel to one another and between which the needles 5 are embedded.

FIG. 2a shows an example of a disk 7, equipped with needles 5, in top view on one of the disk surfaces. The disk 7 is made of plastic by injection-molding.

On the surface of the disk 7, several recesses 8 are provided that extend like rays from the inside of the disk up to its outside edge. The angle measured from the disk midpoint between adjacent recesses is in each case 30°. In each of the recesses 8, a needle 5 is inserted and fastened with an adhesive. In the center of the disk, there is a gear rim-like centering projection 10 that projects over the remaining surface of the disk 7. The cavities between the individual teeth of the gear rim simultaneously form a stop 9 for the needles, whose rear ends lie in the cavities. The stops 9 prevent the needles from being able to be shifted under pressure inside the disk.

All needles 5 essentially have a uniform length. They project with a length L over the outside peripheral surface 4 of the roller 3 or each individual disk 7, which corresponds to the desired penetration depth of the needles in the skin to be treated. In the figure, this is illustrated with the dotted line in the lower area of the drawing. This line diagrammatically represents the skin surface. When turning the roller, the latter moves forward over the skin surface, and the needles 5 penetrate the skin surface in succession.

For the rotatable bearing arrangement, each of the disks 7 has a through-hole 13 in the center of the disk. The shaft 14 that is shown in FIG. 1 is pushed through the latter and then fastened to the fork 16.

FIG. 2b shows the disk, shown in FIG. 2a, from the rear side. Instead of the gear rim projection 10, this side of the disk 7 has a correspondingly formed centering recess 11. If several disks, as shown in FIG. 1b, are combined in one roller, the centering projection 10 comes to lie in the centering recess 11 of an adjacent disk. As a result, it is not only ensured that the adjacent disks are centered exactly on one another, but it is also provided that the needles of adjacent disks come to lie in a specific orientation to one another. In the case that is shown, the needles of adjacent disks are arranged in series that run parallel to the longitudinal axis 2 of the roller 3. It is also readily possible, however, to arrange needles of adjacent disks that are staggered with respect to one another.

In the device shown in FIG. 1, nine of the disks shown in FIG. 2 are arranged in parallel to one another. The front closings of the roller 3 form end disks that are distinguished in their design from the middle disks 7. For example, the surfaces of the end disks that point forward are smooth and do not have any recesses or projections. The end disks are also distinguished among one another. One of the end disks has an inside surface configuration that corresponds to that which is shown in FIG. 2a. The gear rim-shaped centering projection 10 engages positively in the corresponding centering device 11 of the adjacent middle disk 7. In addition, the recesses 8 are equipped with needles 5 and form the tenth needle series in the roller depicted in FIG. 1b. Altogether, this roller thus has 120 needles.

The second end disk is depicted in FIG. 3 and referred to by 12. FIG. 3 shows the surface pointing toward the inside of the roller. In the area around the through-hole 13, the end disk 12 has a recess 17. This recess 17 is just large enough to be able to accommodate the gear rim-projection 10 of the adjacent middle disk 7.

The individual disks, which form the roller 3, equipped with needles 5, of the device 1 according to the invention, are fastened to one another by means of an adhesive. Suitably, here, the same adhesive is used that also serves in gluing the needles 5 into the recesses 8 on the disks 7.

1. Device (1) for introducing an active ingredient into the skin, characterized in that it comprises a roller (3) that is mounted to rotate around the longitudinal axis (2) thereof and on whose outside peripheral surface (4) a number of needles (5) project radially outward.

2. Device according to claim 1, wherein the needles (5) are distributed uniformly over the outside peripheral surface (4).

3. Device according to claim 2, wherein the needles (5) are arranged in series parallel to the longitudinal axis (2).

4. Device according to claim 1, wherein the needles (5) with the same length (L) and in particular with a length (L) of 0.12 to 1.5 mm project over the outside peripheral surface (4).

5. Device according to claim 1, wherein the needles (5) are designed to taper to a point outward at least in an area (6) that projects over the outside peripheral surface (4) and, in particular in this point area (6), have a diameter of 0.05 to 0.1 mm, in particular 0.08 mm.

6. Device according to claim 5, wherein the diameter of the needles (5) outside of the point area (6) is between 0.15 and 0.3 mm.

7. Device according to claim 1, wherein the needles (5) consist of high-grade steel.

8. Device according to claim 1, wherein the roller (3) consists of plastic and in particular an injection-molding plastic.

9. Device according to claim 1, wherein the roller (3) consists of parallel disks (7), between which the needles (5) are embedded.

10. Device according to claim 9, wherein the disks (7) have recesses (8) extending outward at least on one of their sides in radial direction to accommodate the needles (5).

11. Device according to claim 10, wherein one stop (9) each for the needles (5) is found on the inside end of the recesses (8).

12. Device according to claim 10, wherein the needles (5) are glued into the recesses (8).

13. Device according to claim 9, wherein at least one centering projection (10), which engages in a corresponding centering recess (11) in the surface of an adjacent disk (7), is provided on one of the disk surfaces.

14. Device according to claim 13, wherein the centering projection (10) serves simultaneously as a stop (9) for at least one of the needles (5) and in particular has the form of a gear rim that projects into the center of the disk (7).

15. Device according to claim 1, wherein adjacent recesses (8) of a disk (7) lie in straight lines that form an angle of 15° or 30° from the midpoint of the disk (7).

16. Device according to claim 1, wherein the needles (5) or recesses (8) of adjacent disks (7) are arranged in gaps.

17. Device according to claim 9, wherein the front sides of the roller (3) are closed with end disks (12).

18. Device according to claim 1, wherein the roller (3) is mounted to rotate on a fork (16) that is provided with a handle (15) and shifts the stress on the latter in the longitudinal direction.

19. Device according to claim 18, wherein the roller (3) has a through-hole (13) that comprises the longitudinal axis (2) and in which a shaft (14), to which the fork (16) is fastened, is inserted.

20. Process for introducing an active ingredient into the skin, wherein the active ingredient or a mixture that contains the active ingredient is applied on the skin and then the device according to claim 1 is guided over the skin such that the needles (5) penetrate the skin.
